(19) United States
(12) Patent Application Publication
(10) Pub. No.: US 2004/0213765 A1
(43) Pub. Date: Oct. 28, 2004

(54) Use of bacterial phage associated lytic enzymes to prevent food poisoning

(76) Inventors: Vincent Fischetti, West Hempstead, NY (US); Lawrence Loomis, Columbia, MD (US); David Trudil, Reisterstown, MD (US)

Correspondence Address: New Horizons Diagnostics, Inc., 9110 Red Branch Road, Columbia, MD, 21045-2014, US

(21) Appl. No.: 09/910,940

(22) Filed: Jul. 13, 2001

(51) Int. Cl. A61K 38/43; A23K 1/165; A23K 1/17

(52) U.S. Cl. 424/93.6; 424/94.1; 424/442

(57) ABSTRACT

The present invention discloses a method and composition for the treatment of bacterial contamination of food by the use of a phage associated lysing enzyme, preferably blended with an appropriate carrier. The method for treating food stuffs comprises treating the food stuffs with an anti-infection agent comprising an effective amount of at least one lytic enzyme produced by a bacteria infected with a bacteriophage specific for the bacteria. Additionally, chimeric lytic enzymes shuffled lytic enzymes, and holin proteins, either alone or in combination, may be used to treat or prevent bacterial contamination of foodstuffs. The lytic enzyme can be used for the treatment or prevention of various strains of Staphylococcus, Streptococcus, Listeria, Salmonella, E. coli, Campylobacter, Pseudomonas, Brucella, other bacteria, and any combination thereof. Feed for livestock, poultry and beef in slaughterhouses, canned and bottled goods, salad bars, and eggs are just some of the food items that can be treated with at least one lytic enzyme to reduce the risk of food contamination by bacteria.

This application claims benefit less than 35 USC 120 of U.S. application Ser. No. 09/704,148, filed Nov. 2, 2001.

### BACKGROUND OF THE INVENTION

1. Field of the Invention

The present invention discloses a method and composition to prevent food poisoning by the use of phage associated lysing enzymes and modified versions of the lysing enzymes.

2. Description of the Prior Art

Bacterial contamination is a serious problem in the food industry. It is estimated that each year, thousands ofpeople in the United States, and millions worldwide die of ingesting contaminated food and drinking water. As the population of the world continues to grow, and as cities become more crowded and agricultural land becomes more scarce, there has been an increase in the amount of food that must be processed and the amount of intensive farming which must be done, thereby resulting in the increase of food contamination. In the United States, the number of chickens infected by Salmonella, beef infected with E. coli, and the number of rivers, streams and bays infected by farm run off, has been rising each of the last several years.

In the past, antibiotics have been used to treat various bacterial infections. The work of Selman Waksman in the introduction and production of Streptomycetes and Dr. Fleming's discovery of penicillin, as well as the work of numerous others in the field of antibiotics are well known. Over the years, there have been additions and chemical modifications to the “basic” antibiotics in attempts to make them more powerful, or to treat people allergic to these antibiotics.

These antibiotics have been incorporated into feedstuffs for cattle, chickens, and turkeys to prevent illnesses in the animals before they get to the slaughter houses. However, as more antibiotics have been prescribed or used at an ever increasing rate for a variety of illnesses, increasing numbers of bacteria have developed a resistance to antibiotics. Larger doses of stronger antibiotics are now being used to treat ever more resistant strains of bacteria. Multiple antibiotic resistant bacteria have consequently developed. The use of more antibiotics and the number of bacteria showing resistance has led to increasing the amount of time that the antibiotics need to be used. Broad, nonspecific antibiotics, some of which have detrimental effects on the animals, are now being used more frequently.

Once these animals are slaughtered and arrive on the dinner tables of millions of people world wide, there remain chemical remnants of the antibiotics in the food. As many individuals are allergic to antibiotics, they suffer numerous medical problems when the food is ingested, such as diarrhea, headaches, stomach aches, hives, etc. Turkeys are notorious for retaining a high level of antibiotics.

The introduction of infectious agents also occurs in meat processing plants. The “fecal baths” in chicken processing plants and the bacterial contamination in beef processing plants, particularly in the production of hamburger meat, remain notorious in the food industry. Of course, bacterial contamination of food can be found along other locations of the food processing chain, such as at salad bars, where individual customers often handle the food and then place it back on the table, thereby infecting the salad with Listeria, Salmonella, E. coli, Staphylococcus, or Streptococcus. Chicken eggs are often contaminated with Salmonella. Numerous bacteria can infect the water with which food is prepared. Scientists, consumers, and grocers are finding that fish are frequently contaminated with bacteria. This problem has increased as waste from the suburbs and from agribusinesses and industrial farms washes into the Chesapeake Bay.

Additionally, other food stuffs can suffer from contamination. Salad bars are often unsanitary. Canned and bottled goods are also food stuffs which frequently become contaminated, either before or after the containers are opened by consumers.

Attempts have been made to treat bacterial diseases by the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses aprocedure of improved dental hygiene which comprises introducing into the mouth bacteriophages parasitic to bacteria which possess the property of readily adhering to the salivary pellicle.

It is to be noted that the direct introduction of bacteriophages into an animal to prevent or fight diseases has certain drawbacks. Specifically, the bacteria must be in the right growth phase for the phage to attach. Both the bacteria and the phage have to be in the correct and synchronized growth cycles. Additionally, there must be the right number of phages to attach to the bacteria; if there are too many or too few phages, there will be either no attachment or no production of the lysing enzyme. The phage must also be active enough. The phages are also inhibited by many substances including bacterial debris from the organism it is going to attack. Further complicating the direct use of bacteriophages to treat bacterial infections is the possibility of immunological reactions, rendering the phage nonfunctional. Another problem is the mutation of the receptor on the bacterial surface, preventing bacteriophage attachments.

Consequently, others have explored the use of other safer and more effective means to treat and prevent bacterial infections.

U.S. Pat. No. 5,604,109 (Fischetti et al. and incorporated by reference) relates to the rapid detection of Group A streptococci in clinical specimens, through the enzymatic digestion by a semi-purified Group C streptococcal phage associated lysin enzyme. The present invention is based upon the discovery that phage associated lytic enzymes specific for bacteria infected with a specific phage can effectively and efficiently break down the cell wall of the bacterium in question. At the same time, in most if not all cases, the semipurified enzyme is lacking in mammalian cell receptors and therefore tends to be less destructive to mammalian proteins and tissues when present during the digestion of the bacterial cell wall.

U.S. Pat. No. 6,017,528 (Fischetti, et. al.), U.S. Pat. No. 5,997,862 (Fischetti et al.), and U.S. Pat. No. 5,985,271 (Fischetti et al.) disclose composition and use of an oral delivery mode, such as a candy, chewing gum, lozenge, troche, tablet, a powder, an aerosol, a liquid or a liquid spray, containing a lysin enzyme produced by group C streptococcal bacteria infected with a C1 bacteriophage for the prophylactic and therapeutic treatment of Streptococcal A throat infections, commonly known as strep throat. This is the lysin enzyme of U.S. Pat. No. 5,604,109 (incorporated by reference).

The same general technique used to produce and purify a lysin enzyme shown in U.S. Pat. No. 5,604,109 may be used to manufacture other lytic enzymes produced by bacteria infected with a bacteriophage specific for that bacteria. Depending on the bacteria, there may be variations in the growth media and conditions.

U.S. Pat. No. 6,056,954 (Fischetti et al.) discloses a method for the prophylactic and therapeutic treatment of bacterial infections which comprises the treatment of an individual with an effective amount of a lytic enzyme composition specific for the infecting bacteria, with the lytic enzyme comprising an effective amount of at least one lytic enzyme, and a carrier for delivering said a lytic enzyme. This method and composition can be used for the treatment of upper respiratory infections, skin infections, wounds, and burns, vaginal infections, eye infections, intestinal disorders and dental problems.

U.S. Pat. No. 6,056,955 (Fischetti et al.) discloses the topical treatment of streptococcal infections.

The use of phage associated lytic enzymes produced by the infection of a bacteria with a bacteria specific phage has numerous advantages for the treatment of diseases. As the phage are targeted for specific bacteria, the lytic enzymes generally do not interfere with normal flora. Also, lytic phages primarily attack cell wall structures, which are not affected by plasmid variation. The actions of the lytic enzymes are fast and do not depend on bacterial growth. Additionally, lytic enzymes can be directed to the mucosal lining, where, in residence, they will be able to kill colonizing bacteria.

However, no one has used a phage associated enzyme to prevent or treat bacterial infections in the food chain.

### SUMMARY OF THE INVENTION

The present invention discloses the use of bacterial phage associated lytic enzymes, to prevent or halt bacterial infections or contamination of food, food products, livestock, chicken, or anywhere else in the food chain. More specifically, a lytic enzyme produced by a bacteria infected with a bacteriophage specific for the bacteria may be used. The lytic enzyme produced may be a product of genetic manipulation yielding a shuffled lytic enzyme or a chimeric lytic enzyme.

The method for obtaining and purifying the lytic enzyme produced by bacteria infected with the bacteriophage is known in the art. Some recent evidence suggests that the phage enzyme that lyses the streptococcus organism may actually be a bacterial enzyme that is used to construct the cell wall and the phage. While replicating in the bacterium, a phage gene product may cause the upregulation or derepression of the bacterial enzyme(s) for the purpose of releasing the bacteriophage. These bacterial enzymes may be tightly regulated by the bacterial cell and are used by the bacteria for the construction and assembly of the cell wall.

The use of these lytic enzymes to prevent bacterial growth in food, however, has not been explored. Consequently, the present invention discloses the extraction and use of a variety of bacterial phage associated lytic enzymes, holin proteins, chimeric enzymes, and shuffled enzymes for the treatment or prevention of bacterial infections of food stuffs in the food processing chain. More specifically, the present invention discloses the use of both unmodified and modified versions of bacterial phage associated lytic enzymes, which may include unmodified lytic enzymes, chimeric lytic enzymes, and shuffled lytic enzymes to prevent bacterial infections of food, food products, livestock, chicken, or anything else in the food chain. The term “modified” shall refer to theose enzymes which are shuffled or chimeric forms of the lytic enzyme.

The use of phage associated lytic enzymes produced by the infection of bacteria with bacteria specific phage has numerous advantages for the treatment of specific bacteria. As the phage are targeted for specific bacteria, the lytic enzymes do not interfere with normal flora. Also, lytic phages primarily attack cell wall structures which are not affected by plasmid variation. The actions of the lytic enzymes are fast and do not depend on bacterial growth.

These phage induced lytic enzymes are useful in killing a variety of bacterial pathogens including those involved in food contamination such as but not limited to Salmonella, Streptococcus, Pseudomonas.

The present invention discloses the extraction and use of a variety of bacterial phage associated holin proteins, chimeric lytic enzymes, and shuffled lytic enzymes, in addition to lytic enzymes, for increased efficiency for the treatment of a wide variety of bacterial contaminants. More specifically, the present invention provides a pharmaceutical composition comprising at least one bacteria-associated phage enzyme that is isolated from one or more bacteria species and includes phage lytic and/or holin enzymes. In one embodiment, the lytic enzymes or holin proteins, including their isozymes, analogs, or variants, are used in a modified form. In another embodiment the lytic enzymes or holin proteins, including their isozymes, analogs, or variants, are used in a combination of natural and modified forms. The modified forms of lytic enzymes and holin proteins are made synthetically by chemical synthesis and/or DNA recombinant techniques. and, more preferably, the enzymes are made synthetically by chimerization and/or shuffling.

According to one embodiment, the composition includes one or more natural lytic enzyme produced by the bacterial organism, after being infected with a particular bacteriophage, for prophylactic or therapeutic treatment. Preferably, the composition contains combinations of one or more natural lytic enzyme and one or more chimeric or shuffled lytic enzymes.

Chimeric lytic enzymes are lytic enzymes which are a combination of two or more lytic enzymes having two or more active sites such that the chimeric enzyme can act independently on the same or different molecules. This will allow for potentially treating two or more different bacterial infections at the same time.

Holin proteins produce holes in the cell membrane. More specifically, holins form lethal membrane lesions that terminate respiration. Like the lytic enzymes, the holin proteins are coded for and carried by a genome. In fact, it is quite common for the genetic code for the holin to be found next to or even within the code for the lytic enzyme in the phage. Most holin sequences are short, and overall, hydrophobic in nature, with a highly hydrophilic carboxy-terminal domain. In many cases, the putative holin is encoded on a different reading frame within the enzymatically active domain of the phage. In other cases, the holin is encoded on the DNA next to or close to the DNA coding for the phage. The holin is frequently synthesized during the late stage of phage infection and found in the cytoplasmic membrane where it causes membrane lesions.

Holin proteins can be grouped into two general classes based on primary structure analysis. Class I holins are usually 95 residues or longer and may have three potential transmembrane domains. Class II holins are usually smaller, at approximately 65-95 residues, and the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all of the phages. Holins have been shown to be present or suggested for among others, lactococcal bacteriophage Tuc2009, lactococcal φLC3, pneumococcal bacteriophage EJ-1, Lactobacillus gasseri bacteriophage φadh, Staphylococcus aureus bacteriophage Twort, Listeria monocytogenes bacteriophages, pneumococcal phage Cp-1, Bacillus subtillis phage Φ29, Lactobacillus delbrueckki bacteriophage LL-H lysin, and bacteriophage φ11 of Staphylococcus aureus. (Loessner, et al., Journal of Bacteriology, August 1999, p. 4452-4460).

It should be noted that some in the scientific community believe that holins are enzymes, and not just proteins.

Shuffled enzymes are enzymes in which the genes, gene products, or peptides for more than one related phage enzyme have been randomly cleaved and reassembled into a more active or specific enzyme. Shuffled oligonucleotides, peptides or peptide fragment molecules are then selected or screened to identify a molecule having a desired functional property. This method is described, for example, in Stemmer, U.S. Pat. No. 6,132,970. (Method of shuffling polynucleotides); Kauffman, U.S. Pat. No. 5,976,862 (Evolution via Condon-based Synthesis) and Huse, U.S. Pat. No. 5,808,022 (Direct Codon Synthesis). The contents of these patents are incorporated herein by reference.

Shuffling is used to create an enzyme 10 to 100 fold more active than the template. The template enzyme is selected among different varieties of lysin or holin enzymes. The shuffled enzyme constitutes, for example, one or more binding domains and one or more catalytic domains. Each of the binding or catalytic domains is derived from the same or different phage or phage enzyme. The shuffled domains are either oligonucleotide based molecules, as gene or gene products, that either alone or in combination with other genes or gene products are translatable into a peptide fragment, or they are peptide based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable of translation into a peptide.

All isozymes, variants or analogs ofthe bacterial-associated phage enzymes ofthe invention, whether natural or modified, are encompassed and included within the scope of the invention.

More specifically, the sequence of enzymes when purified can be determined by conventional techniques, and rearrangements of primary structures can be achieved by state of the art techniques, such as shuffling, to increase the activity and stability of the enzyme(s). Shuffling also allows for combination enzymes (“chimeric enzymes”) to have more than one activity.

The creation, purification, and isolation of chimeric, shuffled and lytic enzymes, and holin proteins are well known to those skilled in the art. In particular, U.S. Pat. No. 6,132,970 (Stemmer) (incorporated herein by reference) discloses a number of new techniques, and modifications of more established procedures, for the creation of these enzymes. The proposed invention utilizes these techniques and applies them for the enhancement of specifically noted phage associated lytic enzymes. The technique for isolating lysin enzymes found in U.S. Pat. No. 6,056,954 (also incorporated herein by reference) may be applied to other phage associated lytic enzymes. Similarly, other state of the art techniques may be used to isolate lytic enzymes.

To produce shuffled lytic enzymes, genes of phage lytic enzymes will be shuffled to select for enzymes with more marrow or broad specificity, depending on the specific application. By using this method, a single enzyme may be developed that has, for example, specificity for both S. pyogenes and S. pneumoniae.

In a preferred embodiment of the invention, shuffled enzymes are used to treat bacterial infections, thereby increasing the speed and efficiency with which the bacteria are killed.

Chimeric lytic enzymes are enzymes which are a combination of two or more enzymes having two or more active sites such that the chimeric enzyme can act independently on the same or different molecules. This will allow for potentially treating two or more different bacterial infections at the same time. Chimeric lytic enzymes may also be used to treat one bacterial infection by cleaving the cell wall in more than one location. Chimeric lytic enzymes can be produced by fusing the binding domain of one enzyme with the catalytic domain of a second enzyme, thus taking advantage of the efficiency of cleavage of an enzyme with a highly active catalytic domain, and combining it to a binding domain for a specific bacterium creating a more efficient enzyme for killing the bacterium.

A number of chimeric lytic enzymes have been produced and studied. Gene E-L, a chimeric lysis constructed from bacteriophages phi X174 and MS2 lysis protein's E and L, respectively, was subjected to internal deletions to create a series of new E-L clones with altered lysis or killing properties. The lytic activities of the parental genes E, L, E-L, and the internal truncated forms of E-L were previously investigated to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 July 1, 164(1); 159-67.

Similarly, in another experiment an active chimeric cell wall lytic enzyme (TSL) has been constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumococcal cell walls.

A preferred embodiment ofthis invention discloses the use of chimeric lytic enzymes to treat two infectious bacteria at the same time, or to cleave the cell wall of a bacterium in two different locations.

In another embodiment of the invention, holin proteins are used in conjunction with the lytic enzymes to accelerate the speed and efficiency at which the bacteria are killed. Holin proteins may also be in the form of chimeric and/or shuffled proteins. Holins may also be used alone in the treatment of bacterial infections.

Holins proteins usually work on the cytoplasmic membrane to create a hole allowing the lytic enzyme access to the peptidoglycan causing lysis. In some cases, for example with gram-negative bacteria, it may be necessarily to add holin proteins to the lytic enzyme, thereby allowing the holin to create a hole in the outer membrane of the gram-negative bacteria, enabling the lytic enzyme access to the peptidoglycan externally.

In addition, in some cases, it may be necessary to add EDTA or detergents to destroy or destabilize the outer membrane of gram-negative bacteria to allow the lytic enzymes access to the peptidoglycan.

It should be noted that in this patent, for the sake of simplicity, chimeric lytic enzymes and shuffled lytic enzymes may be referred to as modified versions of the lytic enzyme.

It is an object ofthe invention to use phage associated lytic enzymes, holins, chimeric lytic enzymes, shuffled lytic enzymes, or combinations thereof to prevent bacterial contamination of food.

In one embodiment of the invention, at least one phage associated lytic enzyme, holin, chimeric lytic enzymes, shuffled lytic enzyme, or combinations thereof are used to treat food stuffs used to feed cattle, chickens, sheep or other live stock.

In another embodiment ofthe invention salad bars are treated with at least one phage associated lytic enzyme, holin protein, chimeric lytic enzyme, shuffled lytic enzyme, or combinations thereof to prevent the growth or to kill contaminating bacteria.

In yet another embodiment of the invention, eggs are treated with at least one phage associated lytic enzyme, holin protein, chimeric lytic enzyme, shuffled lytic enzyme, or combinations thereof to prevent or kill Salmonella and other bacterial contamination.

The invention also proposes spraying or incorporating at least one phage associated lytic enzyme, holin protein, chimeric lytic enzymes shuffled lytic enzyme, or combinations thereof in ground beef to kill or prevent the growth of E. coli.

Another embodiment ofthe invention proposes spraying at least one phage associated lytic enzyme, holin protein, chimeric lytic enzyme, shuffled lytic enzyme, or combinations thereof over beef and chicken carcasses in slaughterhouses, or bathing the beef and chicken carcasses in a pool containing the appropriate phage associated lytic enzymes.

The phage associated lytic enzymes, holin proteins, chimeric enzymes, shuffled enzymes, or combinations thereof can also be added to canned goods to kill or prevent the growth of certain bacteria, and to bottled goods to prevent food from turning rancid.

Additionally, phage associated lytic enzymes, holins, chimeric enzymes, shuffled enzymes, or combinations thereof can be added to bottled water to prevent the growth of bacteria. In any and all of these uses, a holin protein may be used alone or in combination with the lytic enzymes (modified or unmodified) to lyse the cells. The holin protein may be shuffled or chimeric.

The invention (which incorporates U.S. Pat. No. 5,604,109 in its entirety by reference) uses an enzyme produced by the bacterial organism after being infected with a particular bacteriophage to lyse specific bacteria. The present invention is based upon the discovery that lytic enzymes specific for bacteria infected with a specific phage can effectively and efficiently break down the cell wall of the bacterium in question. At the same time, the semipurified enzyme is lacking in proteolytic enzymatic activity and therefore non-destructive to mammalian proteins and tissues when present during the digestion of the bacterial cell wall.

In one embodiment ofthe invention, the treatment of a variety of food contaminants, including Staphylococcus aureus, E. Coli, Salmonella, Listeria, Campylobacter, and Brucella are disclosed. The phage associated lytic enzymes, holins, chimeric enzymes, shuffled enzymes, or combinations thereof are put in a variety of carriers and administered according to need.

In one embodiment ofthe invention, a feed stock comprises at least one lytic enzyme, holins, chimeric enzyme, shuffled enzyme, or combinations thereof produced by bacteria infected with a bacteriophage specific for said bacteria.

More specifically, in one embodiment of the invention, the feed stock of cattle is treated with at least one phage associated lytic enzyme, holins, chimeric enzyme, shuffled enzyme, or combinations thereof.

In another embodiment of the invention, the feed stock of chickens is treated with at least one phage associated lytic enzyme, holins, chimeric enzymes, shuffled enzymes, or combinations thereof.

In yet another embodiment of the invention, the feed stock of turkeys is treated with at least one phage associated lytic enzyme, holins, chimeric enzyme, shuffled enzyme, or combinations thereof Similarly, the feed stock of hogs is treated with at least one phage associated lytic enzyme, holins, chimeric enzyme, shuffled enzyme, or combinations thereof.

In another embodiment ofthe invention, eggs are dipped in or sprayed with a solution or liquid containing at least one phage associated lytic enzyme, holins, chimeric enzyme, shuffled enzyme, or combinations thereof.

In another embodiment of the invention, a salad bar contains salad treated with at least one lytic enzyme, holins, chimeric enzyme, shuffled enzyme, or combinations thereof

In yet another embodiment of invention, a bacterial resistant ground beef contains at least one lytic enzyme produced by bacteria infected with a bacteriophage specific for that bacteria.

Again, in all of these uses, at least one holin protein may be used alone or in combination with the phage associated lytic enzyme.

### BRIEF DESCRIPTION OF THE DRAWINGS

FIG. 1 is an electron micrograph of group A streptococci treated with lysin showing the collapse of the cell wall and the cell contents pouring out;

FIG. 2 is a chart showing the lethality of the lysin enzyme for the killing of bacteria on chicken parts;

FIG. 3 is a graph for the killing of S. pneumoniae (#DCC 1490) serotype 14 with PAL at various dilutions;

FIG. 4 is a graph showing the the decrease of bacterial titer within 30 seconds after addition of 100 U Pal phage enzyme;

FIG. 5 is a series of graphs showing the decrease of the Bacterial titer with 30 seconds after the addition of 100, 1,000, and 10,000 U Pal Lytic Enzyme; and

FIG. 6 is a series of graphs showing the decrease of bacterial titer within 30 seconds after addition of different amounts of U Pal.

### DETAILED DESCRIPTION OF THE INVENTION

Lytic enzymes and their modified forms can be used along the entire food processing chain either in place of antibiotics or to prevent the dangerous infectious bacteria from growing where antibiotics have not, or cannot, be used.

The method for treating food stuffs comprises treating the food stuffs with an anti-infection agent comprising an effective amount of at least one lytic enzyme produced by a bacterium infected with a bacteriophage specific for the bacteria, holins, chimeric enzyme, shuffled enzyme, or combinations thereof. More specifically, the lytic enzyme may be either supplemented by chimeric and/or shuffled lytic enzymes, or may be itself a chimeric and/or shuffled lytic enzyme. Similarly, a holin protein may be included, which may also be a chimeric and/or shuffled protein. The lytic enzyme, shuffled lytic enzyme, chimeric lytic enzyme, and/or holins is preferably in an environment having a pH which allows for activity of the enzyme. In a preferred embodiment of the invention, the holin enzyme may be used in conjunction with the administration of the lytic enzyme, shuffled lytic enzyme, and/or chimeric lytic enzyme. The holins may be in its “natural” state, may be a shuffled holin protein or may be a chimeric.

Additionally, compositions ofthis invention include one or more bacteria-associated phage enzymes, including isozymes, analogs, or variants thereof, in a natural or modified form. The modified form of the enzyme, for example, shuffled and/or chimeric enzymes, is produced enzymatically by chemical synthesis and/or DNA recombination technology.

It should be understood that bacteriophage lytic enzyme are enzymes that specifically cleave bonds that are present in the peptidoglycan of bacterial cells. Since the bacterial cell wall peptiodglycan is highly conserved among all bacteria, there are only a few bonds to be cleaved to disrupt the cell wall. Enzymes that cleave these bonds are muramidases, glucosaminidases, endopeptidases, or N-acetyl-muramoyl L alanine amidases (hereinafter referred to as amidases). The majority of reported phage enzymes are either muramidases or amidases, and there have been no reports of bacteriophage glucosaminidases. Fischetti et al (1974) reported that the C1 streptococcal phage lysin enzyme was an amidase. Garcia et al (1987, 1990) reported that the Cp-1 lysin from a S pneumoniae phage was a muramidase. Caldentey and Bamford (1992) reported that a lytic enzyme from the phi 6 Pseudomonas phage was an endopeptidase, splitting the peptide bridge formed by meso-diaminopimilic acid and D-alanine. The E. coli T1 and T6 phage lytic enzymes are amidases as is the lytic enzyme from Listeria phage (ply) (Loessner et al, 1996).

There are a large number of phages which will attach to specific bacteria and produce enzymes which will lyse that particular bacteria. The following are a list of bacteriophages and bacteria for which they are specific:

Streptococci

Pseudomonas

Pneumococci

Salmonella

Staphylococci

Shigella

Haemophilus

Listeria

Mycobacteria

Vibrio

Corynebacteria

Bacillus

Spirochete

Myxococcus

Burkholderia

Brucella

Yersinia

Clostridium

Campylobacter

Neisseria

Actinomycetes

Agrobacterium

Alcaligenes

Clostridium

Coryneforms

Cyanobacteria

Enterobacteria

Lactobacillus

Lactoctococcus

Micrococcus

Pasteurella

Rhizobium

Xanthomonas

Bdellovibrio

mollicutes

Chlamydia

Spiroplasma

Caulobacter

Various phages which can be used to infect these bacteria and create the lytic enzyme include:

| BACTERIA | PHAGE(S) |
|---|---|
| Actinomycetes | A1-Dat, Bir, M1, MSP8, P-a-1, R1, R2, SV2, VP5, PhiC |
| | φ31C, φUW21, φ115-A, φ150A, 119, SK1, 108/016 |
| Aeromonas | 29, 37, 43, 51, 59.1 |
| Altermonas | PM2 |
| Bacillus | AP50, φNS11, BLE, Ipy-1, MP15, mor1, PBP1, |
| | SPP1, Spbb, type F, alpha, φ105, 1A, II, Spy-2, |
| | SST, G, MP13, PBS1, SP3, SP8, SP10, |
| | SP15, SP50 |
| Bdellovibrio | MAC-1, MAC-1′, MAC-2, MAC-4, |
| | MAC-4′, MAC-5, MAC-7 |
| Caulobacter | φCb2, φCb4, φCb5, φCb8r, φCb9, φCB12r, φCb23r, |
| | φCP2, φCP18, φCr14, φCr28, PP7, φCb2, φCb4, φCb5, |
| | φCb8r, φCb9, φCB12r, φCb23r, φCP2, φCP18, φCr14, |
| | φCr28, PP7 |
| Chlamydia | Chp-1 |
| Clostridium | F1, HM7, HM3, CEB, |
| Coliform | AE2, dA, Ec9, f1, fd, HR, M13, ZG/2, ZJ/2 |
| Coryneforms | Arp, BL3, CONX, MT, Beta, A8010, A19 |
| Cyanobacteria | S-2L, S-4L, N1, AS-1, S-6(L) |
| Enterobacter | C-2, If1, If2, Ike, I2-2, PR64FS, SF, tf-1, PRD1, |
| | H-19J, B6, B7, C-1, C2, Jersey, ZG/3A, T5, ViII, b4, |
| | chi, Beccles, tu, PRR1, 7s, C-1, c2, fcan, folac, |
| | Ialpha, M, pilhalpha, R23, R34, ZG/1, ZIK/1, ZJ/1, |
| | ZL/3, ZS/3, alpha15, f2, fr, FC3-9, |
| | K19, Mu, 01, P2, ViI, φ92, |
| | 121, 16-19, 9266, C16, DdVI, PST, SMB, SMP2, a1, |
| | 3, 3T+, 9/0, 11F, 50, 66F, 5845, 8893, M11, QB, |
| | ST, TW18, VK, FI, ID2, fr, f2, |
| Listeria | H387, 2389, 2671, 2685, 4211 |
| Micrococcus | N1, N5 |
| Mycobacterium | Lacticola, Leo, R1-Myb, 13 |
| Pasteurella | C-2, 32, AU |
| Pseudomonas | Phi6, Pf1, Pf2, Pf3, D3, Kf1, M6, PS4, SD1, PB-1, |
| | PP8, PS17, nKZ, nW-14, n1, 12S, |
| Staphyloccous | 3A, B11-M15, 77, 107, 187, 2848A, Twort |
| Streptococcus | A25, A25 PE1, A25 VD13, A25 omega8, A25 24 |
| Steptococcus A | |
| Vibrio | OXN-52P, VP-3, VP5, VP11, alpha3alpha, IV, kappa, |
| | 06N-22-P, VP1, x29, II, nt-1, |
| Xanthomonas | Cf, Cf1t, Xf, Xf2, XP5 |

There are numerous other phages infecting these and other bacteria. The bacteriophages are normally grouped into family, genus and species, including Genus Chlamydiamicrovirus, Genus Bdellomicrovirus, Genus Spiromicrovirus, Genus Microvirus, Genus Microvirus, Genus Levivirus, Genus Allolevivirus, and other genuses.

The DNA coding of these phages and other phages may be altered to allow the recombinant enzyme to attack one cell wall at more than two locations, to allow the recombinant enzyme to cleave the cell wall of more than one species of bacteria, to allow the recombinant enzyme to attack other bacteria, or any combinations thereof. The type and number of alterations to the recombinant bacteriophage produced enzyme are incalculable

It should be noted that holin proteins are particularly useful when phage associated lytic enzymes are used to treat gram negative bacteria. More specifically, in some instances, it may be necessary to add holin proteins to the lytic enzyme, thereby allowing the holin protein to create a hole in the outer membrane of gram negative bacteria, thereby enabling the lytic enzyme access to the peptidoglycan externally. If the addition of holin protein alone does not work, it may be preferable to add EDTA or detergents to destabilize the outer membrane of gram negative bacteria to allow the lytic enzymes access to the peptidoglycan. Additionally, it may be possible to use holin enzymes alone to lyse some enzymes.

In the preferred embodiment of the invention, lytic enzymes, chimeric lytic enzymes, shuffled lytic enzymes, holin proteins, and EDTA may be mixed together for optimal use under battlefield conditions.

For example, infection of the Hemophilus bacteria by Bacteriophage HP1 (a member of the P2-like phage family with strong similarities to coliphages P2 and 186, and some similarity to the retrophage Ec67) produces a lytic enzyme capable of lysing the bacteria. The lytic enzyme for Streptococcus pneumoniae, previously identified as an N-acetyl-muramoyl-L-alanine amidase, is produced by the infecting Streptococcus pneumoniae with the Pal bacteriophage. The anti-bacterial agent can contain either or both of the lytic enzymes produced by these two bacteria, and may contain other lytic enzymes for other bacteria.

The lytic enzyme, a holin protein, chimeric enzyme, shuffled enzyme, or combinations thereof can be used for the treatment or prevention of various strains of Staphylococcus, Streptococcus, Listeria, Salmonella, E. coli, Campylobacter, Pseudomonas, Brucella, other bacteria, and any combination thereof.

This lytic enzyme may be either supplemented by chimeric and/or shuffled lytic enzyme, or may be itself a chimeric and/or shuffled lytic enzyme. Similarly, a holin protein may be included, which may also be chimeric and/or shuffled.

Antibiotics in animal feed can be readily replaced with lytic enzymes, holins, chimeric lytic enzymes, shuffled lytic enzymes, or combinations thereof. The lytic enzymes and their variations can be for a variety of bacteria which are found in animal feed. When applied to the feed, the lytic enzymes and their variations will kill the bacteria for which the lytic enzyme is specific. When the animal ingests the feed, there will be no adverse effects of the lytic enzyme to the animal. The protection afforded to the feed will be transferred to the animal, except for those lytic enzymes and modified forms digested in the animal's digestive tract.

Animal feeds can be either “dry” or “wet.” It is quite common that the animal feed is in the form of a thick slurry. In those instances, prior to feeding the animals, at least one lytic enzyme, a holin protein, chimeric lytic enzyme, shuffled lytic enzyme, or combinations thereof is added and mixed into the slurry. The enzyme(s) can be lyophilized or dehydrated. However, the lytic enzyme(s) added can also be in a carrier. Alternatively, during the processing ofthe feed stock, the feed can be bathed in a lytic enzyme bath, prior to packaging or prior to use. The feed can also be sprayed after it is placed in the feeding pen or trough.

The carrier for the enzyme(s) may be water, an oil immersion, micelles, micelles in water or oil, liposomes, liposome in oil or water, combinations thereof, or any other convenient carrier. The enzyme(s) may be encapsulated in a carbohydrate or starch like structure, or the micelles or liposomes may be encapsulated by a starch or carbohydrate type structure. The carrier may also be in the form of a powder. The taste and texture of the carrier should be pleasing to the animal, so that the animal does not reject the food.

Prior to, or at the time the lytic enzyme(s) a holin protein, chimeric lytic enzyme, shuffled lytic enzyme, or combinations thereof is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. It is to be noted that some enzymes may have optimum pH's outside of this range.

The stabilizing buffer should allow for the optimum activity of the lytic enzyme, a holin protein, chimeric lytic enzyme, shuffled lytic enzyme, or combinations thereof. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

Means of application include, but are not limited to direct, indirect, carrier and special means or any combination of means.

The effective dosage rates or amounts of the lytic enzyme and its modified forms to treat bacteria will depend in part on whether the lytic enzyme, a holin protein, a chimeric lytic enzyme, shuffled lytic enzyme, or combinations thereof will be used therapeutically or prophylactically, the duration of exposure of the recipient to the infectious bacteria, the size and weight of the animal being fed, etc.

It is recognized that the antibiotic administered in the feed is used, in part, preventively, so that when an animal sticks its mouth and nose into the feed trough, it gets a high dosage of antibiotics in its mouth and nasal passages. The dosage of the lytic enzymes, a holin protein, chimeric lytic enzyme, shuffled lytic enzyme, or combinations thereof can be high enough to serve the same function. The concentration of the active units of an enzyme believed to provide for an effective amount or dosage of an enzyme may be in the range of about 100 units/ml to about 500,000 units/ml of fluid in the wet or damp environment of the nasal and oral passages, and possibly in the range of about 100 units/ml to about 100,000 units/ml, and more preferably in the range of about 100 units/ml to about 10,000 units/ml.

Livestock which can be fed feed which has been treated with lytic enzymes, a holin protein, chimeric lytic enzyme, shuffled lytic enzyme, or combinations thereof include, cattle, sheep, chickens, hogs, and any other livestock.

Bacterial infections ofhuman food stuffs often occurs in the slaughterhouse, after the animal has been killed. Chickens on the processing assembly line are often dipped in a water bath, derisively referred to in the industry as “fecal soup” because the internal organs and waste of the dead chickens have fallen into this bath. Consequently, many of the chickens coming off the assembly line are contaminated prior to being packaged and shipped to market. Sometimes the chickens arrive in the grocery store, already spoiled. Other times, the consumer does not thoroughly cook the chicken, at least to a temperature to kill all bacteria present, and consequently the consumer gets food poisoning.

Lytic enzymes, a holin protein, chimeric lytic enzyme, shuffled lytic enzyme, or combinations thereof can be used to help prevent bacterial contamination of the chickens. High levels of these enzymes can be added to the water bath, thereby aiding in the killing of bacteria present. In another preferred method of preventing bacterial contamination and food poisoning, the entire chicken or parts thereof, after coming out of the water bath but prior to being packaged and shipped, can be sprayed with at least one lytic enzyme, a holin protein, chimeric enzyme, shuffled enzyme, or combinations thereof, to kill and prevent the growth of bacteria. It is preferred that the lytic enzyme and its modified forms for use on the chicken be specific for Salmonella or E. coli. The carrier may be water, an oil emulsion, etc. The enzyme(s) may be added in a powder. If added in powder form, it is preferred that a carrier made out of cornstarch, or some other starch be used. The powder may also be a protein powder such as a caseinate, or some other suitable substance

As before, the carrier for the lytic enzyme and its modified forms may be water, an oil immersion, micelles, reverse micelles, micelles in water or oil, liposomes, liposome in oil or water, combinations thereof, or any other convenient carrier. The lytic enzyme and its modified to forms may be encapsulated in a carbohydrate or starch like structure, or the micelles or liposomes may be encapsulated by a starch or carbohydrate type structure. The carrier may also be in the form of a powder. The taste and texture of the carrier should be pleasing to the animal, so that the animal does not reject the food.

Prior to, or at the time the enzyme(s) is (are) put in the carrier system or oral delivery mode, it is preferred that the enzyme(s) be in a stabilizing buffer environment for maintaining a pH range between about 4.0 and about 9.0, more preferably between about 5.5 and about 7.5 and most preferably at about 6.1. It is to be noted that some enzymes may have optimum pH's outside of this range.

Also, as before, the stabilizing buffer should allow for the optimum activity of the lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

Beef and hog carcasses are also subjected to contamination in slaughterhouses. Hence, the carcasses of hogs, beef, and other livestock may also be treated with at least one lytic enzyme, a holin protein, chimeric lytic enzyme, shuffled lytic enzyme, or combinations thereof to kill or prevent bacterial growth. The entire carcass of the animal may be dipped in a solution or liquid containing the lytic enzyme(s), a holin protein, chimeric lytic enzyme, shuffled lytic enzyme, or combinations thereof, or preferably, the carcass may be sprayed with a solution or liquid containing the enzyme. The lytic enzyme or its modified form may also be dusted onto the carcass in a powder, as described above. In a preferred embodiment of the invention, at least one lytic enzyme or its modified form for E. coli, is used. As above, it is preferred that the enzyme be in a carrier, which is buffered for the maximum activation of the lytic enzyme(s) or their modified form and to prevent denaturation of the enzyme(s).

Carcasses are not the only form of meat which suffer from contamination. Ground beef, used in hamburgers, also have a relatively high rate of contamination, compared to the rate of contamination for the rest of the food industry. Each year, a number of people die from eating hamburgers which were undercooked and contaminated, frequently with E. coli bacteria.

Consequently, at least one lytic enzyme or its modified form(s) may be incorporated into the ground meat or ground beef. The enzyme(s) may be added during the grinding of the beef, and may be added as the meat goes through the grinder, or it may be added after the meat is ground. The enzyme(s) may be in a lyophilized or dry form, whereupon the enzyme(s) becomes rehydrated upon contact with the “wet” ground beef. The lyophilized or dry enzymes and their modified forms may be in a powder form, such as in a carbohydrate, cornstarch or protein powder. Alternatively, the enzyme(s) may be in any of the carriers previously described, at the pH also described above. Similarly, holins may be added, either alone or as an addition to the enzyme being used.

Eggs are also subject to contamination, particularly Salmonella contamination. However, the use of lytic enzymes and their modified forms can greatly reduce the risk of Salmonella poisoning. At least one lyophilized lytic enzyme or its modified form may be applied to the shells by dipping or soaking the eggs into a lytic enzyme solution or liquid containing at least one lytic enzyme or its modified form, or by spraying a lytic enzyme solution or liquid containing a lytic enzyme (or its modified forms) onto the shells of the eggs. The lytic enzyme or its modified form(s) may be in a water or oil based solution or liquid, with the enzyme(s) either being directly in the solution or liquid, or being in a micelle, reverse micelles, liposomes, or combinations, thereof. It is preferred that the buffer solution be used prior to the enzyme(s) being put into solution or liquid. In fact, in all uses of the enzyme(s), it is always preferable that the carrier or substance to which the enzyme(s) are to be added is first buffered. The carrier for the lytic enzyme(s) may be also be a powder. The powder, which may be a starch powder, a carbohydrate, or a protein powder, may be sprinkled on the egg. Alternatively, the egg may be rolled in the powder. As before, the holin protein may be added alone or with the lytic enzymes.

Food contamination is often found at salad bars which routinely contain vegetables, fruits, boiled eggs, and cheeses. At salad bars, aside from air-borne contamination, it is regrettably not uncommon for customers to pick up a piece of food, examine it, and return it to the bin from whence it came, thereby contaminating the salad bar with bacteria.

To combat the bacteria, the salad of the salad bar may be sprayed or dusted with at least one lytic enzyme, holin protein, chimeric enzyme, shuffled enzyme, or combinations thereof. In a preferred embodiment, the enzyme, with or without the presence ofthe holin protein, is sprayed on the salad, with the carrier for the lytic enzyme(s) being water. It is preferred that the water is buffered and that the pH is adjusted. Of course, the carrier for the enzymes can be an emulsion, an oil, or any other appropriate substance. The lytic enzyme, holin protein, chimeric enzyme, shuffled enzyme, or combinations thereof can be in a micelle, a liposome, or in a reverse micelle. The enzyme(s) can also be placed in the salad dressing. Lytic enzymes for the bacteria Staphylococcus, Streptococcus, Listeria, Salmonella, E. coli, Campylobacter, Pseudomonas and any combinations thereof can be used to treat the salad bar.

Of course, the surfaces of the salad bar, as well as any other surface that comes in contact with food, can and should also be treated with at least one lytic enzyme, holin protein, to chimeric enzyme, shuffled enzyme, or combinations thereof to destroy any bacteria present on these surfaces. The surfaces should be either sprayed with a solution or emulsion containing at least one enzyme, holin protein, chimeric enzyme, shuffled enzyme, or combinations thereof or the surfaces can be wiped down with a wiping material such as a clean cloth, sponge, or rag which has been saturated with enzymes. The wiping material may be dipped into a buffered solution or liquid containing the enzymes. Alternatively, the wiping material may have the enzymes dehydrated or lyophilized on them, and the surface which is to be wiped is wetted. When the wiping material makes contact with the wet surface, the enzymes are re-hydrolized, and kill the bacteria on the surfaces being wiped.

At least one lytic enzymes, holin proteins, chimeric enzymes, shuffled enzymes, or combinations thereof can also be used in canned and bottled goods to prevent bacterial growth or kill bacteria in these sealed goods. Prior to the sealing of the containers, at least one lytic enzyme, holin protein, chimeric enzyme, shuffled enzyme, or combinations thereof and preferably several enzymes, is (are) added to the bottle or can. The can or bottle is then sealed. Any bacteria present will be killed by the appropriate lytic enzyme, holin protein, chimeric enzyme, shuffled enzyme, or combinations thereof. Some of the enzymes that may be used include the lytic enzymes and their modified version for bacteria Staphylococcus, Streptococcus, Listeria, Salmonella, E. coli, Campylobacter, Pseudomonas. The enzyme(s)and the holin protein may be added in almost any form, from lyophilized form, dehydrated form, in a carrier liquid, protected by micelles or in a liposome, etc. The solution or liquid in which the enzyme is added should be buffered.

It is particularly helpful to add at least one lytic enzyme, holin proteins, chimeric lytic enzymes, shuffled lytic enzyme, or combinations thereof in fruit juices, and to apple juice in particular. When the apples fall on the ground, they pick up E. coli bacteria. Regrettably, apples frequently are not washed before they are turned into cider or juice. Consequently, when the juice is drunk, usually by young children, there is a greater risk of illness. The addition of the lytic enzymes and their modified versions, and preferably the lytic enzyme specific for E. coli, prior to the sealing of the bottle, will diminish the risk of bacterial contamination and illness. The enzymes may be added to other potable liquids, preferably of the non-alcoholic nature. Using the right combination of enzymes could replace Pasteurization.

As with all compositions described in this patent, the composition may further include a bactericidal or bacteriostatic agent as a preservative.

Additionally, the agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comnm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues.

As noted above, the use of the holin lytic enzyme, the chimeric lytic enzyme, and/or the shuffled lytic enzyme, may be accompanied by the use of a “natural” lytic enzyme, which has not been modified by the methods cited in U.S. Pat. No. 6,132,970, or by similar state of the art methods. The phage associated lytic enzyme may be prepared as shown in the following example:

### EXAMPLE 1

Harvesting Phage Associated Lytic Enzyme

Group C streptococcal strain 26RP66 (ATCC #21597) or any other group C streptococcal strain is grown in Todd Hewitt medium at 37.degree. C. to an OD of 0.23 at 650 nm in an 18 mm tube. Group C bacteriophage (C1) (ATCC #21597-B1) at a titer of 5.times.10.sup.6 is added at a ratio of 1 part phage to 4 parts cells. The mixture is allowed to remain at 37.degree. C. for 18 min at which time the infected cells are poured over ice cubes to reduce the temperature of the solution to below 15.degree. C. The infected cells are then harvested in a refrigerated centrifuge and suspended in 1/300th of the original volume in 0.1M phosphate buffer, pH 6.1 containing 5.times.10.sup.−3 M dithiothreitol and 10ug of DNAase. The cells will lyse releasing phage and the lysin enzyme. After centrifugation at 100,000.times.g for 5 hrs to remove most of the cell debris and phage, the enzyme solution is aliquoted and tested for its ability to lyse Group A Streptococci.

The number of units/ml in a lot of enzyme is determined to be the reciprocal of the highest dilution of enzyme required to reduce the OD650 of a suspension of group A streptococci at an OD of 0.3 to 0.15 in 15 minutes. In a typical preparation of enzyme 4.times.10.sup.5 to 4.times.10.sup.6 units are produced in a single 12 liter batch.

Use of the enzyme in an immunodiagnostic assay requires a minimum number of units of lysin enzyme per test depending on the incubation times required. The enzyme is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise an immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bactericidal or bacteriostatic reagent as a preservative. Such preservative can be sodium azide in a concentration from 0.001 percent to 0.1 percent, preferably 0.02 percent.

The preparation ofphage stocks for lysin production is the same procedure described above for the infection of group C streptococcus by phage in the preparation of the lysin enzyme. However, instead of pouring the infected cells over ice, the incubation at 37.degree. C. is continued for a total of 1 hour to allow lysis and release of the phage and the enzyme in the total volume. In order for the phage to be used for subsequent lysin production the residual enzyme must be inactivated or removed to prevent lysis from without ofthe group C cells rather than phage infection.

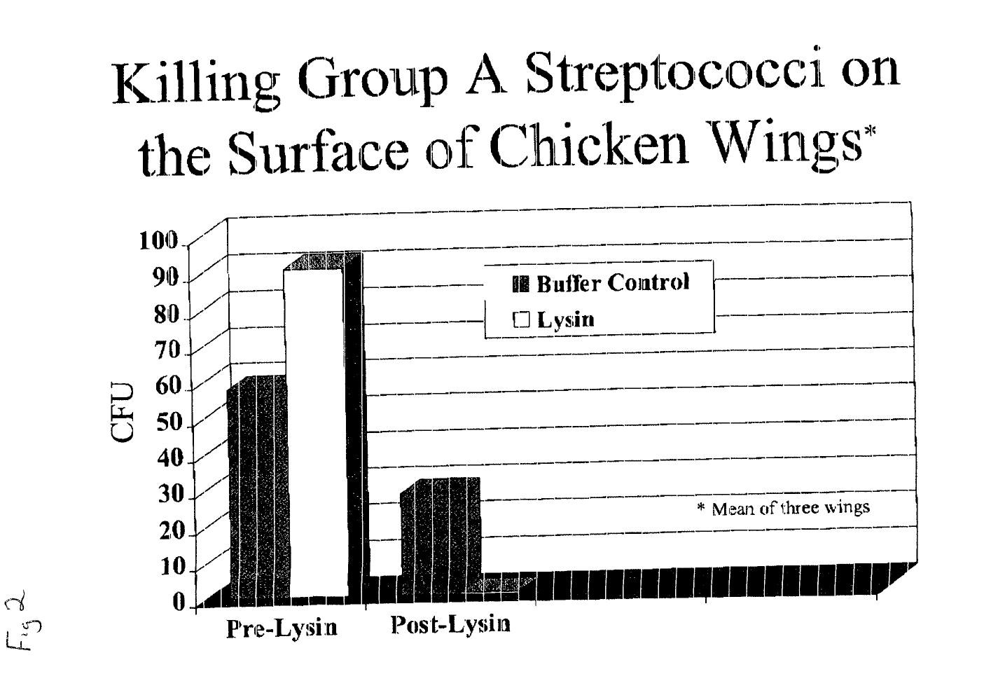

The use of lytic enzymes, including but not limited to holin proteins, chimeric lytic enzymes, shuffled lytic enzymes, and combinations thereof, rapidly lyse the bacterial cell. The thin section electron micrograph of FIG. 1 shows the results of a group A streptococci 1 treated for 15 seconds with lysin. The micrograph (25,000× magnification) shows the cell contents 2 pouring out through a hole 3 created in the cell wall 4 by the lysin enzyme.

The use of lytic enzymes to prevent food poisoning or food contamination is illustrated in the following example.

### EXAMPLE 2

Group A Streptococci (Streptomycin resistant) were grown in Todd-Hewitt broth in mid-log phase and diluted in phosphate buffer (pH 6.1) to yield a final count of 8,400 colony forming units (CFUs) per ml based on plate count. One ml of the streptococcal suspension was spread on the surface of each of six chicken wings and one section of the wing was swabbed with a standard throat swab and the organisms on the swab are spread on the surface of a blood agar plate containing 200 ug/ml of streptomycin (pre treatment).

Three chicken wings were then treated by spraying C1 phage lysin (1.0 ml containing 500 units of enzyme/ml) while a second set of three wings were treated with 1.0 ml of buffer (phosphate buffer pH 6.1). The wings were allowed to sit at room temperature (˜21 degrees Celsius) for ten minutes at which time all wings were again swabbed and spread on blood agar plates containing 200 ug/ml of streptomycin to determine the bacterial counts (post treatment).

As shown in FIG. 1, there was about a 99% decrease in the bacterial count after lysin treatment. The approximately 48% decrease in counts seen in the buffer control may be accounted for by the two fold dilution that occurred after the addition of buffer to the wings.

| | |
|---|---|
| CONTROL | LYSIN |

(Colony Forming Units)

| | | |
|---|---|---|
| Pre Treatment | 58 | 91 |
| Post Treatment | 28 | .6 |

The use of chimeric or shuffled enzymes shows a great improvement as to the properties of the enzyme, as illustrated by the following examples:

### EXAMPLE 3

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 July 1, 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 4

Isolation of the Pal Lytic Enzyme:

Recombinant E.coli DH5 (pMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE 5

Killing Assay:

S. pneumoniae of various serotypes and 8 different viridans streptococi were grown overnight and for most assays diluted and re-grown for 6 h to log phase of growth, pelleted and resupended in 0.9% saline to an OD@620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units.

One unit (U) of Pal was defined as the highest dilution at which Pal decreased the OD of a pneumococcal strain by half in 15 minutes.

### EXAMPLE 6

Production of Chimeric Lytic Enzymes

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 July 1, 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 7

Isolation of the Pal Lytic Enzyme

Recombinant E.coli DH5 (pMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE 8

Killing Assay

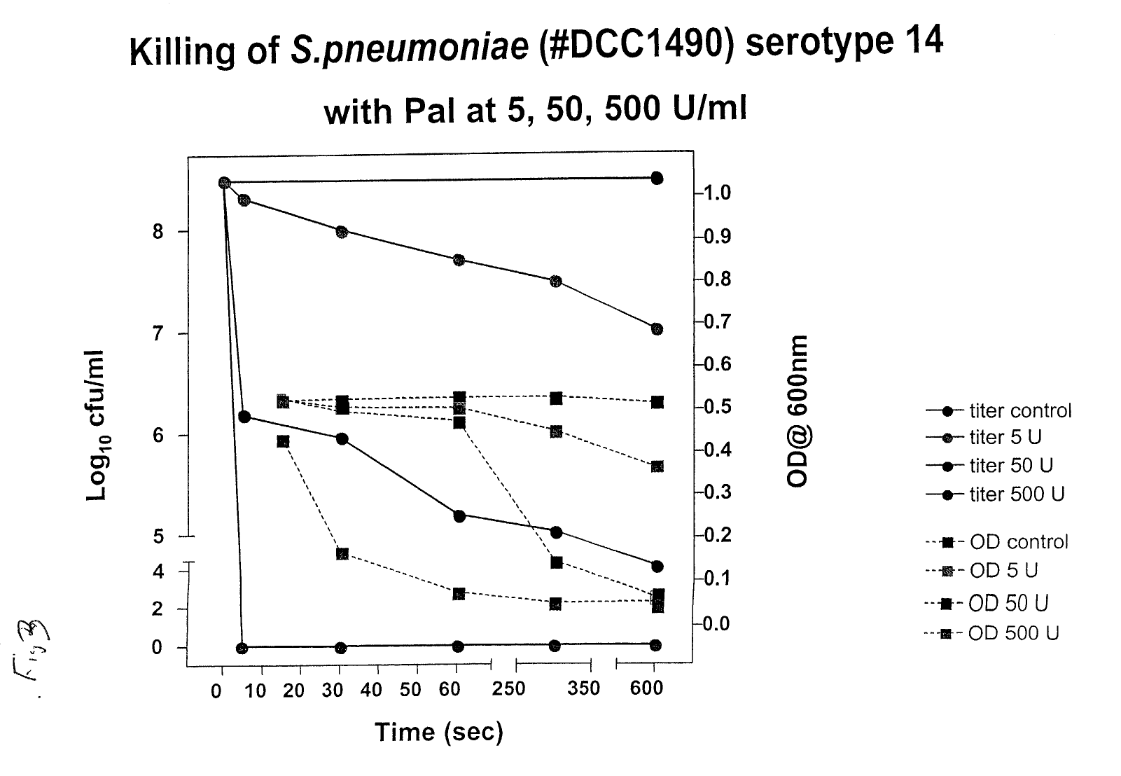

S. pneumoniae of various serotypes and 8 different viridans streptococi were grown overnight and for most assays diluted and re-grown for 6 h to log phase of growth, pelleted and resupended in 0.9% saline to an OD@620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units. One unit (U) of Pal was defined as the highest dilution at which Pal decreased the OD of a pneumococcal strain by half in 15 minutes. The results, (see FIG. 2) show that the viability of Pneumococci dropped more than 8 logs in five seconds after adding the Pal enzyme.

### EXAMPLE 9

Susceptability of Oral Streptoccocci to Pal Enzyme

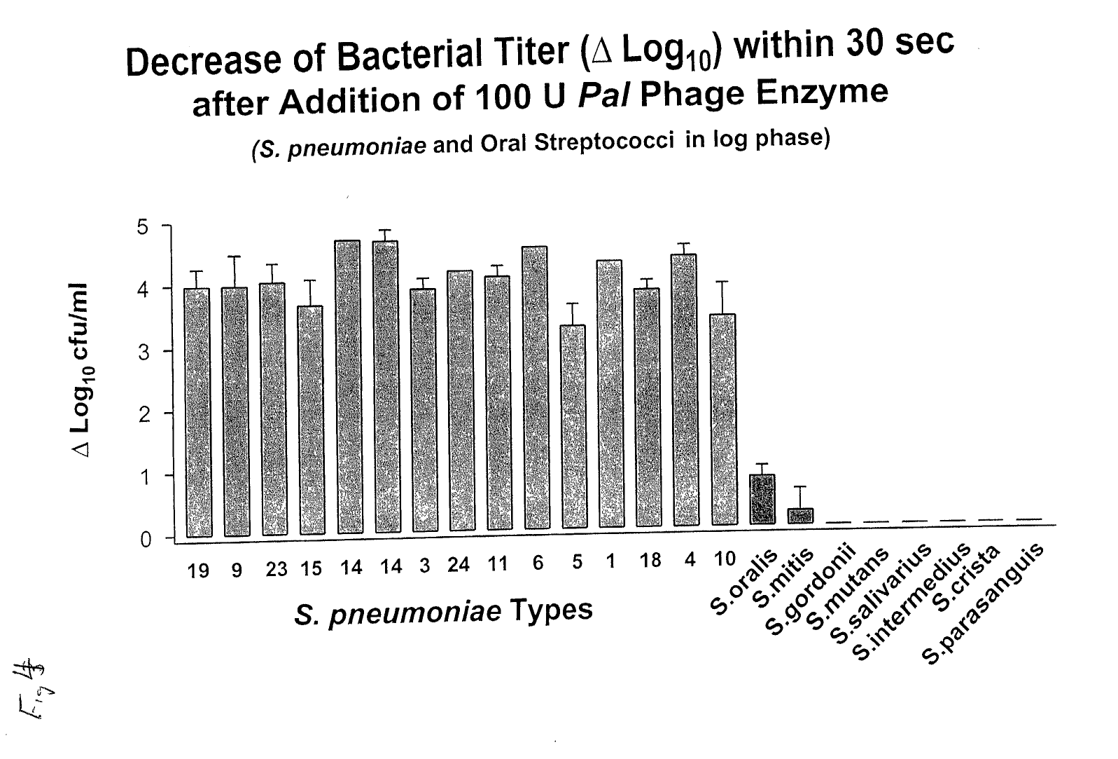

Various serotypes of oral streptoccoci were tested against bacteria-associated lytic enzymes, in particular, the Pal enzyme. A variety of S. pneumoniae type bacteria was also included in the test. Pal enzyme were used at a concentration of 100 U of the purified enzyme. As can be seen in FIG. 3 all S. pneumoniae serotypes are killed (˜4 logs) within the 30 seconds of exposure. Of the oral streptococci tested, only S. oralis and S. mitis show low sensitivity to the Pal enzyme.

### EXAMPLE 10

Susceptability of Stationary Phase Bacteria to Lytic Enzyme

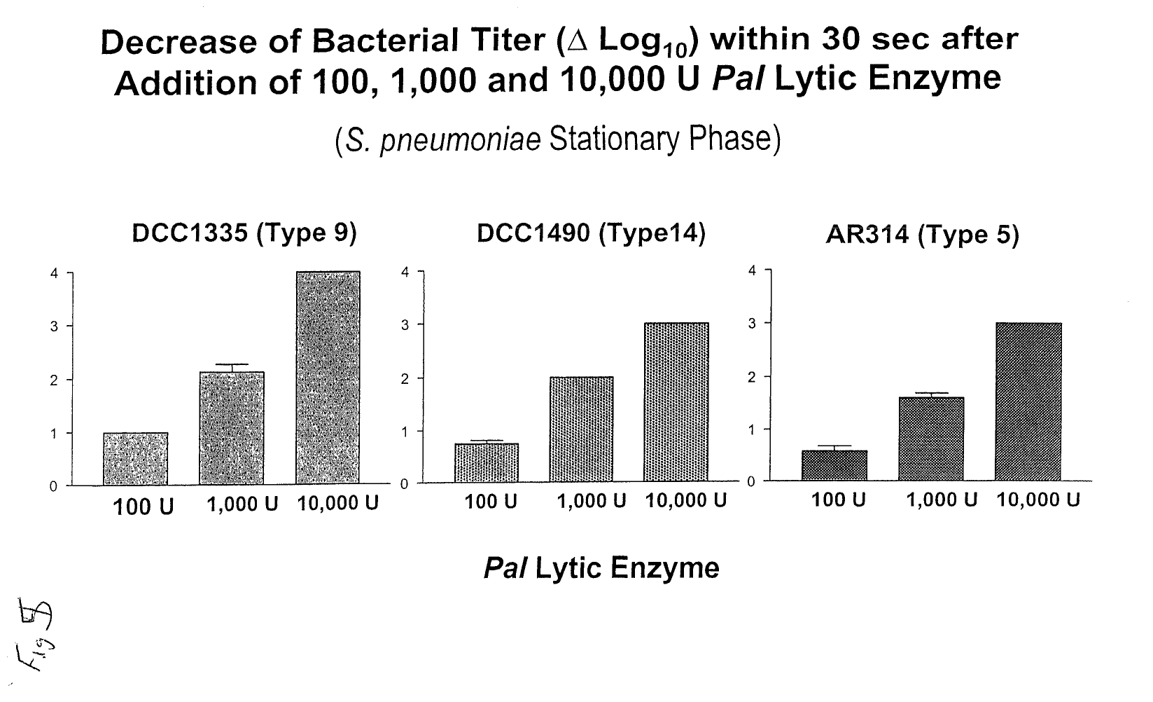

In order to confirm that activity of lytic enzymes are independent of the bacterial grwoth, several serotypes of serotypes of S.pneumoniae at stationary phase of growth were tested against lytic enzymes. In particular, 3 strains of Pal lytic enzyme were used against 3 sereotypes of S. pneumoniae. The results show that that all bacterial strains tested against Pal enzyme were killed in 30 seconds (see FIG. 4). An approximately 2-log drop in viability of the bacteria occurred with 1,000 U of enzyme, as opposed to about 3-4 log drop in the viability with 10,000 units.

### EXAMPLE 11

Effect of Pal Lytic Enzyme on Log-Phase and Stationary Phase Oral Streptococci.

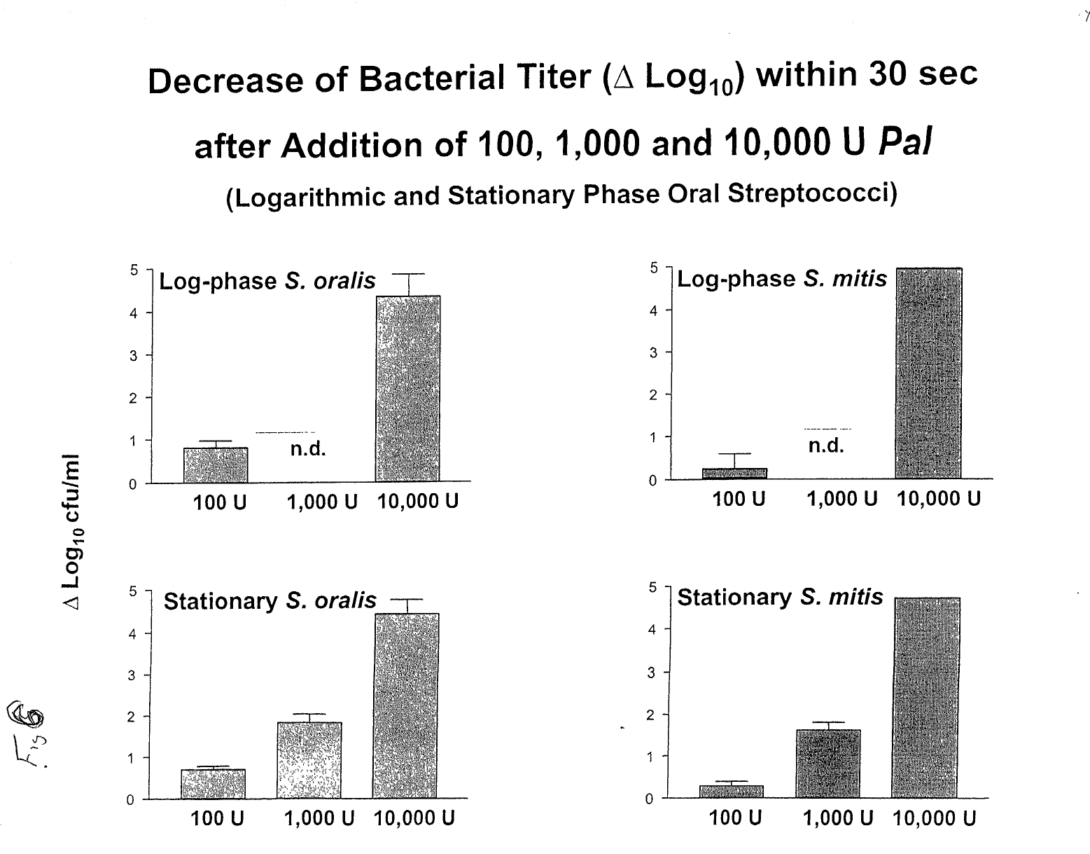

Streptococci oralis and Streptococci mitis in log or stationary phases of growth were treated with different concentrations of the Pal lytic enzyme. Viability was measured after 30 seconds. Results, as shown in FIG. 5, indicate that both bacterial species were equally sensitive to the Pal enzyme in both log or stationary phases of growth.

Many modifications and variations ofthe present invention are possible in light of the above teachings. It is, therefore, to be understood within the scope of the appended claims the invention may be protected otherwise than as specifically described.

Each publication cited herein is incorporated by reference in its entirety.

What we claim is:

1) A method for the prevention of food poisoning, comprising
administering to a food stock:
an effective amount of at least one enzyme produced by a bacteria infected with a bacteriophage specific for said bacteria wherein said at least one enzyme is selected from the group consisting of lytic enzymes, shuffled lytic enzymes, chimeric lytic enzymes, and combinations thereof;
wherein said food stock is selected from the group consisting of live stock feed, eggs, salad bars, beef carcasses, chicken carcasses, food to be canned, and livestock feed.

2) The method of claim 1, wherein said food stock is livestock feed.

3) The method of claim 2, wherein said livestock feed is for the feeding of cattle.

4) The method of claim 2, wherein said livestock feed is for the feeding of chickens.

5) The method of claim 2, wherein said livestock feed is for the feeding of hogs.

6) The method of claim 2, wherein said livestock feed is for the feeding of sheep.

7) The method of claim 2, wherein said livestock feed is dry.

8) The method of claim 2, wherein said livestock feed is a slurry.

9) The method of claim 1, further comprising delivering said at least one enzyme in a carrier suitable for delivering said at least one said enzyme.

10) The method according to claim 1, wherein said at least one enzyme is specific for at least one strain of Pseudomonas.

11) The method according to claim 1, wherein said at least one enzyme is specific for Streptococcus pneumoniae.

12) The method according to claim 1, wherein said at least one enzyme is specific for Streptococcus fasciae

13) The method according to claim 1, wherein said at least one enzyme is specific for at least one strain of Listeria.

14) The method according to claim 1, wherein said at least one enzyme is specific for at least one strain of Salmonella.

15) The method according to claim 1, wherein said at least one enzyme is specific for at least one strain of E. coli.

16) The method according to claim 1, wherein said at least one enzyme is specific for at least one strain of Campylobacter.

17) The method according to claim 1, wherein said at least one enzyme is specific for at least one strain of Pseudomonas.

18) The method according to claim 1, wherein said at least one enzyme is specific for Streptococcus mutans.

19) The method according to claim 1, wherein said at least one enzyme is specific for Mycobacterium tuberculosis.

20) The method according to claim 1, wherein said at least one enzyme is specific for at least one strain of Streptococcus.

21) The method according to claim 9, wherein said carrier is selected from the group consisting of water, oil, micelles, inverted micelles, liposomes, starches, carbohydrates, and combinations thereof.

22) The method according to claim 1, wherein said at least one enzyme is in an environment having a pH which allows for activity of said at least one enzyme.

23) The method according to claim 1, wherein said at least one enzyme is in a buffer that maintains pH of the composition at a range between about 4.0 and about 9.0.

24) The method according to claim 23, wherein said buffer maintains the pH ofthe composition at the range of between about 5.5 and about 7.5.

25) The method according to claim 23, wherein said buffer comprises a reducing agent.

26) The method according to claim 25, wherein said reducing agent is dithiothreitol.

27) The method according to claim 23, wherein said buffer comprises a metal chelating reagent.

28) The method according to claim 27, wherein said metal chelating reagent is ethylenediaminetetraacetic disodium salt.

29) The method according to claim 23, wherein said buffer is a citrate-phosphate buffer.

30) The method according to claim 1, further comprising a bactericidal or bacteriostatic agent as a preservative.

31) The method according to claim 1, wherein said at least one enzyme is present in an amount ranging from about 100 to about 500,000 units per milliliter.

32) The method according to claim 31, wherein said at least one enzyme is present in an amount ranging from about 1,000 units to about 100,000 units per milliliter.

33) The method according to claim 32, wherein said at least one enzyme is present in an amount ranging from about 10,000 units to about 100,000 units per milliliter.

34) The method according to claim 1, wherein said food stock is a salad bar, comprised of salad.

35) The method according to claim 9, wherein said at least one enzyme is administered by spraying said at least one enzyme onto said salad.

36) The method according to claim 35, wherein said carrier for spraying said at least one enzyme onto said salad is selected from the group consisting of water and an oil based mixture.

37) The method according to claim 35, wherein said at least one enzyme is contained in a protecting structure selected from the group consisting of a micelle, reverse micelle, liposome, and combinations.

38) The method according to claim 34, wherein said at least one enzyme is administered by dusting said at least one enzyme onto said salad.

39) The method according to claim 9, wherein said at least one said enzyme is applied to carcasses of animals in a slaughterhouse processing plant.

40) The method according to claim 38, wherein said animals are selected from the group consisting of cattle, hogs, sheep, and chickens.

41) The method according to claim 39, further comprising administering said at least one to enzyme by dipping said carcasses of said animals into a liquid containing said at least one enzyme.

42) The method according to claim 39, further comprising administering said at least one enzyme by dusting said at least one enzyme onto the carcasses of said animals in the slaughterhouse processing plant.

43) The method according to claim 9, wherein said at least one enyzme is added during grinding of ground meat.

44) The method according to claim 43, wherein said ground meat is ground beef.

45) The method according to claim 43, wherein said carrier carrying said at least one enzyme is a liquid carrier.

46) The method according to claim 43, wherein said carrier carrying said at least one enzyme is in the form of a powder, said powder being selected from the group selected from a carbohydrate powder, a cornstarch powder, and a protein powder.

47) The method according to claim 9, wherein said at least one enzyme is added to ground meat after said meat is ground.

48) The method according to claim 9, wherein said food stock is at least one egg.

49) The method according to claim 48, wherein said carrier is a liquid, and said at least one egg is dipped into said liquid.

50) The method according to claim 48, wherein said carrier is a liquid, and said liquid containing said at least one enzyme is sprayed on said at least one egg.

51) The method according to claim 48, wherein said carrier is a powder, and said powder containing said at least one enzyme is sprinkled on said at least one egg.

52) The method according to claim 48, wherein said carrier is a powder, and said egg is rolled in said powder.

53) The method according to claim 1, wherein said at least one enzyme is added to a closed container containing said food stock, said at least one enzyme being added prior to said container being closed during food processing.

54) The method according to claim 53, wherein said closed container is a bottle.

55) The method according to claim 53, wherein said closed container is a can.

56) The method according to claim 53, wherein said at least one enzyme is lyophilized.

57) The method according to claim 53, further comprising a carrier suitable for delivering said at least one enzyme.

58) The method according to claim 57, wherein said carrier is selected from the group consisting of water, emulsion, and a solution.

59) The method according to claim 53, wherein said at least one enzyme is protected by a structure selected from the group consisting of micelles, liposomes, and inverted micelles.

60) The method according to claim 53, further comprising a buffer that maintains pH of the composition at a range between about 4.0 and about 9.0.

61) A method for the treatment and prevention of food contamination, comprising:
administering to a surface where food resides an effective amount of at least one enzyme selected from the group consisting of lytic enzymes, shuffled lytic enzymes, chimeric lytic enzymes, and combinations thereof, and combinations thereof.

62) The method according to claim 61, further comprising a carrier suitable for delivering said at least one lytic enzyme.

63) The method according to claim 62, wherein said carrier is selected from the group consisting of water, emulsion, and a solution.

64) The method according to claim 62, wherein said carrier is applied to said surface with a cloth.

65) The method according to claim 62, wherein said carrier is applied to said surface with a sponge.

66) The method according to claim 62, wherein said carrier is sprayed on said surface.

67) A method for treating animal feed to prevent or treat bacterial contamination, comprising, administering to said animal feed an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled enzymes, chimeric enzymes, holin enzymes, and combinations thereof.

68) The method of claim 67, wherein said animal feed is for an animal selected from the group consisting of cattle, chickens, hogs and sheep.

69) The method of claim 67, wherein said animal feed is dry.

70) The method of claim 67, wherein said animal feed is a slurry.

71) The method of claim 67, further comprising delivering said at least one lytic enzyme in a carrier suitable for delivering said at least one lytic enzyme.

72) A bacterial resistant animal feed comprising:
an animal feed; and
an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof.

73) The bacterial resistant animal feed of claim 72, further comprising a carrier, wherein said at least one enzyme is in said carrier.

74) The method for treating salad bars to prevent or treat bacterial contamination, comprising:
administering to said salad of said salad bar an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof.

75) The method of claim 74, further comprising delivering said at least one enzyme in a carrier suitable for delivering said at least one lytic enzyme.

76) A bacterial resistant salad bar comprising:
salad in a salad display in a public area; and
an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof.

77) The bacterial resistant salad bar of claim 76, further comprising a carrier, wherein said at least one enzyme is in said carrier.

78) A method for treating carcasses of animals to prevent food poisoning, comprising:
administering to said carcasses of said animals an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled enzymes, chimeric enzymes, holin enzymes, and combinations thereof to said carcasses of said animals.

79) The method according to claim 78, wherein said animals are selected from the group consisting of cattle, hogs, sheep, and chickens.

80) The method according to claim 78, further comprising dipping said carcasses of said animals into a liquid containing said at least one enzyme.

81) The method according to claim 80, further comprising dusting said at least one enzyme onto the carcasses of said animals in the slaughterhouse processing plant.

82) A method for treating ground meat to prevent food poisoning, comprising:
administering to said ground meat an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof. to said carcasses of said animals.

83) The method of claim 82, wherein said at least one enzyme is added during the grinding of ground meat.

84) The method of claim 81, wherein said enzyme is in a carrier suitable for delivering said at least one enzyme.

85) The method of claim 82, wherein said carrier is selected is selected from the group consisting of water, oil, micelles, inverted micelles, liposomes, starches, carbohydrates, or combinations thereof.

86) A bacteria resistant ground meat, comprising:
ground meat; and an
effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof.

87) The bacteria resistant ground meat of claim 86, wherein said ground meat is ground beef.

88) The bacteria resistant ground meat of claim 86, wherein said bacteria for which said enzyme is specific is E. coli.

89) The bacteria resistant ground meat of claim 86, further comprising a carrier, wherein said at least one enzyme is in said carrier.

90) The bacteria resistant ground meat of claim 89, wherein said carrier is selected from the group consisting ofwater, oil, micelles, inverted micelles, liposomes, starches, carbohydrates and combinations thereof.

91) A method for treating eggs to prevent food poisoning, comprising administering to shells of said eggs an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof. to said carcasses of said animals.

92) The method of claim 91, further comprising a carrier suitable for delivering said at least one enzyme to the shells of said egg.

93) The method of claim 92, wherein said eggs are dipped in a solution comprising said at least one enzyme.

94) The method of claim 92, wherein said shells of said eggs are dusted with a carrier comprising said at least one enzyme.

95) The method of claim 91, wherein said carrier is selected from the group consisting of water, oil, micelles, inverted micelles, liposomes, starches, carbohydrates, and combinations thereof.

96) A method for reducing bacterial infections of sealed food containers, comprising administering to said food containers before they are sealed an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof. to said carcasses of said animals.

97) The method of claim 96, wherein said sealed food container is a bottle.

98) The method of claim 96, wherein said sealed food container is a can.

99) The method of claim 96, further comprising delivering said enzyme in a carrier suitable for delivering said enzyme.

100) The method of claim 99, wherein said carrier is selected from the group consisting of a micelle, reverse micelle, liposome, and combinations thereof.

101) The method according to claim 96, wherein the enzyme is in an environment having a pH which allows for activity of said enzyme.

102) The method according to claim 101, wherein said enzyme is in a buffer that maintains pH of the composition at a range between about 4.0 and about 9.0.

103) The method according to claim 103, wherein said buffer maintains the pH of the composition at the range of between about 5.5 and about 7.5.

104) The method according to claim 102, wherein said buffer comprises a reducing agent.

105) The method according to claim 104, wherein said reducing agent is dithiothreitol.

106) The method according to claim 102, wherein said buffer comprises a metal chelating reagent.

107) The method according to claim 106, wherein said metal chelating reagent is ethylenediaminetetraacetic disodium salt.

108) The method according to claim 102, wherein said buffer is a citrate-phosphate buffer.

109) The method according to claim 96, further comprising a bactericidal or bacteriostatic agent as a preservative.

110) The method according to claim 96, wherein said at least one enzyme is present in an amount ranging from about 100 to about 500,000 units per milliliter.

111) The method according to claim 110, wherein said at least one enzyme is present in an amount ranging from about 1,000 units to about 100,000 units per milliliter.

112) The method according to claim 111, wherein said at least one enzyme is present in an amount ranging from about 10,000 units to about 100,000 units per milliliter.

113) A method for reducing bacterial infections of liquids, comprising administering to said liquids an effective amount of at least one enzyme selected from the group consisting of at least one lytic enzyme produced by a bacteria infected with a bacteriophage specific for said bacteria, at least one modified version of said at least one lytic enzyme, and combinations thereof, wherein said modified version of said at least one lytic enzyme is selected from the group consisting of shuffled lytic enzymes, chimeric lytic enzymes, holin enzymes, and combinations thereof.

114) The method of claim 113, wherein said liquid is in a bottle.

115) The method of claim 113, wherein said liquid is in a can.

116) The method of claim 113, further comprising delivering said enzyme in a carrier suitable for delivering said enzyme.

117) The method of claim 116, wherein said carrier is selected from the group consisting of a micelle, reverse micelle, liposome, and combinations thereof.

118) The method according to claim 113, wherein the enzyme is in an environment having a pH which allows for activity of said enzyme.

119) The method according to claim 118, wherein said enzyme is in a buffer that maintains pH of the composition at a range between about 4.0 and about 9.0.

120) The method according to claim 119, wherein said buffer maintains the pH of the composition at the range of between about 5.5 and about 7.5.

121) The method according to claim 119, wherein said buffer comprises a reducing agent.

122) The method according to claim 121, wherein said reducing agent is dithiothreitol.

123) The method according to claim 119, wherein said buffer comprises a metal chelating reagent.

124) The method according to claim 123, wherein said metal chelating reagent is ethylenediaminetetraacetic disodium salt.

125) The method according to claim 119, wherein said buffer is a citrate-phosphate buffer.

126) The method according to claim 113, further comprising a bactericidal or bacteriostatic agent as a preservative.

127) The method according to claim 113, wherein said at least one enzyme is present in an amount ranging from about 100 to about 500,000 units per milliliter.

128) The method according to claim 127, wherein said at least one enzyme is present in an amount ranging from about 1,000 units to about 100,000 units per milliliter.

129) The method according to claim 128, wherein said at least one enzyme is present in an amount ranging from about 10,000 units to about 100,000 units per milliliter.
